(19) United States
(12) Patent Application Publication
(10) Pub. No.: US 2023/0087473 A1
(43) Pub. Date: Mar. 23, 2023

(54) COMPOSITION OF SUSPOEMULSION FORMULATION OF ANTHELMINTIC DRUGS WITH ESSENTIAL OILS FOR NASO-PULMONARY ADMINISTRATION

(71) Applicants: HEMANT N. JOSHI, Parsippany, NJ (US); Neelam Sharma, Morris Plains, NJ (US)

(72) Inventors: HEMANT N. JOSHI, Parsippany, NJ (US); Neelam Sharma, Morris Plains, NJ (US)

(21) Appl. No.: 17/481,730

(22) Filed: Sep. 22, 2021

(51) Int. Cl. A61K 9/107; A61K 45/06; A61K 9/00; A61K 31/4184; A61K 31/166; A61K 31/7048

(52) U.S. Cl. CPC A61K 9/107; A61K 45/06; A61K 9/0043; A61K 31/4184; A61K 31/166; A61K 31/7048

(57) ABSTRACT

A suspo-emulsion dosage form of anthelmintic drug such as Ivermectin or niclosamide with essential oils was proposed to be used for administration to the nasal cavity and lungs. The suspo-emulsion formulation is to be administered using a suitable medical device. The composition may be used to treat or prevent viral infections such as Covid-19.

### FIELD OF INVENTION

This patent application relates to nasal and pulmonary delivery of medicinal agents to treat variety of conditions. The system includes a medical device and a composition suitable to treat ailments in the pulmonary system. We are claiming the compositions and the suspo-emulsion dosage form with a combination formulation of an allopathic drug(s) with a natural drug(s) for naso-pulmonary administration. This application claims a priority benefit to U.S. Provisional Application No. 63/107,624, filed on Oct. 30, 2020. The aforementioned application is incorporated herein in the references.

### BACKGROUND OF THE INVENTION

The present invention relates to a novel suspo-emulsion drug delivery system of a pharmaceutical composition comprising of an allopathic drug(s) and a natural drug(s) for naso-pulmonary administration. It also describes its method of preparation of the same. The patent specifically describes a combination of anthelmintic drug(s) and essential oils as actives in a suspo-emulsion dosage form.

Ivermectin, an anthelmintic drug, is an old drug commonly used to treat many types of parasite infestations. This includes head lice, scabies, river blindness (onchocerciasis), trichiasis, ascariasis, and lymphatic filariasis. It can be taken by mouth or applied to the skin for external infestations. Ivermectin has been shown to be effective against viruses such as Zika, dengue. West Nile, Hendra, Newcastle, Chikungunya, HIV type 1 and Corona viruses in in-vitro and in-vivo studies (Formiga et al. and Heidary et an.

Many essential oils have been commonly used to treat congestion, sinus problems, etc. These essential oils have been reported to possess anti-bacterial, anti-viral and anti-inflammatory properties (Gandhi et al). Some of the commonly used oils are—Tea tree oil, lemongrass oil, menthol, eucalyptus oil, clove oil, oregano oil, cinnamon oil etc.

Covid 19 is believed to reside in the sinuses for the first two days before travelling down deep in lungs. Being volatile, a nasal administration of essential oils delivers these oils to sinuses (Frontal, Ethmoid, Maxillary and Sphenoid). This helps to relieve congestion in sinuses. U.S. Pat. No. 9,655,939 (Reeve R. A. and Michael, T. P. J., May 2017) taught to control microbes in the sino-nasal tract. Essential oils also get delivered deep in the lungs and may alleviate the symptoms due to viral infection, inflammation etc. M. Asif et al. predicted the application of essential oils in controlling Covid 19.

This new composition is in the form of a suspo-emulsion, containing an essential oil(s), more specifically the essential oils with antiviral, antibacterial and anti-inflammatory properties and an anthelmintic drug(s). Anthelmintic drugs such as ivermectin, albendazole, mebendazole, thiabendazole, fenbendazole, triclabendazole, flubendazole, abamectin, salicylanilide, nitazoxanide, niclosamide, octadepsipeptides, monepantel, spiroindoles etc. can be incorporated in this dosage form. We prefer to use more specifically Ivermectin.

The purpose of the present invention is to develop a new composition and a novel dosage form having properties of essential oil and antiviral activity of anthelmintic drug(s). Ivermectin has demonstrated broad spectrum antiviral activity against both DNA and RNA viruses. Ivermectin, an anti-helminthic drug, is also being proposed for treatment and prevention of COVID-19. Other anthelmintic drugs such as niclosamide can be added instead of ivermectin.

Ivermectin is an approved broad spectrum anti-helminthic agent, which has previously been demonstrated to have anti-viral activity against both DNA and RNA viruses including HIV-1, dengue, West Nile virus. Venezuelan equine encephalitis virus (VEEV) and influenza virus (Formiga et al). Ivermectin inhibits the host importin protein imp α/β1 heterodimer involved mainly in the transport of SARS-CoV-2 nucleocapsid protein to nucleus, which plays a vital role in viral pathogenesis. As a consequence of this inhibition, the drug is proposed to be beneficial in limiting the disease spread and severity, and has the potential to be used as an effective therapeutic and prophylactic agent against SARS-CoV-2 (Formiga et al). Caly et al demonstrated the inhibitory role of ivermectin on replication of SARS-COV-2 in an in-vitro study.

Combination drug dosage forms are becoming common in recent years. Multiple drugs, when combined, may have complimentary activities. Some adjunct therapies can also reduce the side-effects of the main drug. This is a logic behind using the combination of anthelmintic compounds and essential oils.

Anthelmintic drugs, in general, have low water solubility. One can solubilize water-insoluble compounds by adding surface active agents or surfactants. However, surface active agents in high concentration my produce undesirable side-effects on the naso-pulmonary mucosa. It was therefore decided to focus on developing a nano-suspension or micro-suspension formulations and minimize the usage and concentration of surface-active agents. Essential oils are not miscible in water and oil-in-water formulation is the best way to deliver such essential oils in vivo. This is the rationale behind developing a suspo-emulsion dosage form—a combination of a suspension and an emulsion.

Ivermectin dose and pharmacokinetics—Ivermectin tablets are currently available in the market. However, sufficient amount of ivermectin does not enter in the respiratory system (Schmith et al and Jermain et al.). Thus, to obtain a sufficient ivermectin concentration in the respiratory tract, it will be important to administer the drug directly in the respiratory system. Tay et al. described a case of nasal myiasis due to Musca Domenstica in a 97-year-old Peruvian farmer. Due to nasal irrigation with ivermectin solution, the patient self-expulsed approximately 50 larvae within 15 minutes. Niclosamide, another anthelmintic drug, has very poor water solubility and shows very low bioavailability. Recent studies have indicated that niclosamide may have broad clinical applications for the treatment of diseases other than those caused by parasites. These diseases and symptoms may include cancer, bacterial and viral infection, metabolic diseases such as Type II diabetes, NASH and NAFLD, artery constriction, endometriosis, neuropathic pain, rheumatoid arthritis, sclerodermatous graft-versus-host disease, and systemic sclerosis (W. Chen et al., Rejinold et al., Xu et al. and Brunaugh et al.).

Essential oils are known for their antimicrobial, antiviral and anti-inflammatory properties. Essential oils (EOs) have been screened against several pathogenic viruses, including influenza and other respiratory viral infections. Owing to their lipophilic nature, EOs are advocated to penetrate viral membranes easily leading to membrane disruption. Moreover, EOs contain multiple active phytochemicals that can act synergistically on multiple stages of viral replication and also induce positive effects on host respiratory system including bronchodilation and mucus lysis. At present, only computer-aided docking and few in vitro studies are available, which show anti-SARC-CoV-2 activities of EOs.

Thus, the pharmaceutical composition according to the invention is in the form of a suspo-emulsion whose formula contains therapeutically effective or prophylactic quantities of anthelmintic drug(s) and EOs as active ingredients and a suitable mixture of excipients. Many drugs have limited water solubility and a suspension formulation becomes a potential option. EOs are oils and therefore, can be incorporated into aqueous formulation as an emulsion. Thus, a combination formulation of anthelmintic drugs, having low aqueous solubility, with an EO results into a suspo-emulsion drug delivery system. Various kinds of primary packaging and medical devices can be used for these formulations. These can be nasal sprays, inhalers, nebulizers, metered dose devices etc. For the purpose of this patent, the naso-pulmonary administration can be achieved using these devices. The drug should be delivered right from nasal cavity and up to deep in lungs to the alveoli.

The novelty of this delivery system resides in the selection of actives, suspo-emulsion dosage form, and the particle size distribution so that the actives are delivered to the entire naso-pulmonary area including nose, sinuses, nasal cavity, trachea, and lungs. A conversion to Nano- or micro-particles increases the surface area exponentially resulting in significantly higher dissolution rate of the drug. Both EOs and anthelmintic drug such as Ivermectin, niclosamide have anti-SARC-CoV-2 activities (Caly et al. and Rejinold et al.) and thus, this suspo-emulsion formulation is unique not only in composition but also as a novel dosage form and can be an effective approach in combating the viral pandemic. Another novelty of this dosage form resides in the selection of an allopathic drug(s) and a natural EO(s). Most allopathic medicinal products contain only a single active drug substance.

### SUMMARY OF THE INVENTION

The present invention proposes a composition of an anthelmintic drug(s) and an essential oil(s). The present invention also proposes a novel suspo-emulsion dosage form of this composition. The concept proposes pulmonary suspo-emulsion drug delivery system consisting of pharmacologically effective or therapeutically effective amounts of anthelmintic drugs and essential oils. The terms “therapeutically effective amount or pharmacologically effective amount” as used herein means that the amounts of actives, anthelmintic drugs and essential oils, contained in the proposed compositions and administered by naso-pulmonary route, is of sufficient quantity to achieve desired pharmacological or therapeutic effect(s). One can also use a prophylactic amount of actives in these formulations.

Anthelmintics or anthelminthics are a group of antiparasitic drugs that expel parasitic worms (helminths) and other internal parasites from the body by either stunning or killing them and without causing significant damage to the host. They may also be called vermifuges (those that stun) or vermicides (those that kill). Anthelmintics are used to treat people who are infected by helminths, a condition called helminthiasis. Anthelmintic drugs such as ivermectin, albendazole, mebendazole, thiabendazole, fenbendazole, benzimidazole, praziquantel, triclabendazole, flubendazole, abamectin, salicylanilide, nitazoxanide, niclosamide, octadepsipeptides, monepantel, spiroindoles, oxamniquine, piperazine, morantel, pyrantel, levamisole, tribendimidine, etc. are proposed to be used in this invention. Anthelmintic drugs in this patent are termed as “allopathic medicines”. “Allopathic medicine” in this patent is defined as a “the broad category of science-based medical practice that is also called Western medicine, biomedicine, evidence-based medicine, or modern medicine. Allopathic medicine or drugs includes drug molecules, which are designed using structure-activity correlation studies, and are normally synthesized in the laboratory. It is possible that the original molecule used to develop new drug molecules may be originated from a natural source.

Essential oils such as Tea tree oil, Eucalyptus oil, Lemongrass oil, cinnamon oil etc. are proposed to be used in this invention. For the purpose of this patent application, the EOs are considered to be “Natural Products”, as these are isolated from a natural source—mostly plants. Natural products can be an extract from a plant or it may be an entire plant part. EOs can be extracted from different parts of the plant. such as leaves, flowers, roots, etc. In this patent, essential oils with anti-viral, anti-bacterial and anti-inflammatory activities are used in the formulations. EOs can be used for their therapeutic or prophylactic activities. Prophylactic means EOs may prevent a disease or illness.

An EO is a concentrated hydrophobic liquid containing volatile (easily evaporated at normal temperatures) chemical compounds from plants. EOs are also known as volatile oils, ethereal oils, aethereal, or simply as the oil of the plant from which they were extracted. EOs are often used for aromatherapy, a form of alternative medicine in which healing effects are ascribed to aromatic compounds. Aromatherapy may be useful to induce relaxation, but there is not sufficient science-based evidence that essential oils can effectively treat any condition. Systematic scientific research as in the allopathic system is not normally conducted on EOs to prove their effectiveness to treat or cure any chronic disease or other illnesses due to several reasons. Having said this, many people strongly believe in aromatherapy and use of EOs to treat undesired health conditions. Some EOs, as mentioned in this patent, have been reported to possess empirically anti-viral, anti-bacterial and anti-inflammatory properties. Only those EOs with these desired properties are claimed here.

The novelty of this patent resides in the combination of “allopathic drug molecule(s)” with “Natural products”. The other aspect is a novel drug delivery system, not used normally in the pharmaceutical arena—Suspo-emulsion formulation. It is a combination of a suspension and an emulsion formulations. Also, the dosage form claimed here is specifically for the administration in the respiratory tract—from nose to deep in the lungs. We named it as a naso-pulmonary region in this patent and administration of drug to these regions is termed as “naso-pulmonary administration”.

The present invention proposes stable suspo-emulsion formulations containing a drug, which has limited water solubility, along with EOs. For drugs having limited water solubility, suspension formulation is a good choice. EOs are oils, which are not miscible in water and therefore, can be incorporated into aqueous formulations as an emulsion. We are proposing a combination formulation of anthelmintic drugs, which are in general have limited water solubility, with EOs as a suspo-emulsion drug delivery system. For the purpose of this patent, EOs term refers to Tea tree oil, Eucalyptus oil, Lemongrass oil, cinnamon oil, and Essential oils from Grapefruit, Orange, Tangerine, Mandarine, Lemon, Pine, Peppermint. Lavender, Clove, Thyme, Oregano etc. Other EOs with anti-viral, anti-bacterial and anti-inflammatory activities can also be included. These suspo-emulsion compositions also can contain excipients such as a surfactant(s)/a surface-active agent, antioxidant, humectants etc.

The result of the present invention is a unique composition and dosage form for naso-pulmonary administration. Drug will not only be delivered to the nasal cavity but also to the lungs.

The main objective of these suspo-emulsion composition is to provide a unique composition and dosage form to get nasal and pulmonary drug delivery.

There are various surfactants available and used in pharmaceutical formulations. These are mainly classified as—nonionic, cationic and anionic surfactants. Formulation chemists experienced in the field know the role of surfactants in such formulations. The surfactant(s) must fulfill its role to make a stable nano/micro-suspension and a stable naon/micro-emulsion. However, the formulation will be administered in the naso-pulmonary area and must not be toxic to the cells and produce adverse reactions. Acceptance of the dosage form by the patient is one of key factors in the success of such pharmaceutical formulations. Thus, we are using a minimum quantity of surfactants in these formulations.

The present invention also extends to the process for preparing a pharmaceutical composition, which comprises mixing the EOs with humectant and surfactants etc. followed by water, and finally adding the active followed by suitable processing to produce a suspo-emulsion.

The active principle, anthelmintic drug(s), ranges from 0.01 to 10% by weight, and more preferably between 0.01% to 5% by weight of the formulation.

An individual EO ranges from 0.001 to 1% by weight and more preferably between 0.001 and 0.1% by weight of the formulation according to the composition.

The surfactant(s) is in a proportion of between 0.001 and 3% by weight, preferably between 0.001 and 0.5% by weight of the formulation.

The humectant is in a proportion of between 0.5 and 10% by weight, preferably between 0.5 and 5% by weight of the formulation.

The suspo-emulsion formulation is intended to be delivered in the naso-pulmonary area. Thus, it is important to control the particle size of the suspended drug particles and emulsified EOs. We propose overall D90 value of the particles or globules to be 25 microns or less. D90 or DV (0.9) means that 90% of the total particles are smaller than this size.

Anthelmintic drugs tend to have low bioavailability. Upon oral administration, insufficient blood levels are reached in the respiratory system. If administered by naso-pulmonary route, sufficient local level of the drug can be achieved. One of the key objectives is to achieve a therapeutic or a prophylactic level of anthelmintic drug in the naso-pulmonary area. The other objective is to also administer essential oils with reported antiviral, antibacterial and anti-inflammatory activities locally in the naso-pulmonary area along with the anthelmintic drug. EOs can reach up to the sinuses too.

### DETAILED DESCRIPTION

### Definition of Terms Used

A drug is “a chemical substance used in the treatment, cure, prevention, or diagnosis of disease or used to otherwise enhance physical or mental well-being. Medicament is a medicine, or a substance used in a therapy.

A “medicinal agent” is a pharmaceutical agent that includes drugs, biological products, and medical devices under the regulatory authority of the Food and Drug Administration (FDA). A “medicinal agent” shows pharmacological activity in human subjects and can be “allopathic drug molecules” or “natural drug products”.

Anthelmintic is the term used in this patent describes drugs used to treat infections of animals with parasitic worms. This includes both flat worms, e.g., flukes (trematodes) and tapeworms (cestodes) as well as round worms (nematodes).

“Nasal delivery” is the is a route of administration in which drugs are insufflated or inhaled through the nose. Nasal delivery of drugs circumvents gastrointestinal degradation and hepatic first-pass metabolism of drugs. Nasal delivery can be used to deliver drugs locally or for systemic applications.

“Pulmonary delivery” is a non-invasive means to deliver drugs into nose, nasal cavity and in lungs. In this patent, we used the term “Naso-pulmonary delivery” to indicate delivery to the nasal area and lungs.

“Dosage Form” is the physical form of a dose of a chemical compound used as a drug or medication intended for administration or consumption. A “compound” is a drug substance with intended medicinal action.

“Formulation” is the process in which different chemical substances, including the active drug, are combined to produce a final medicinal product or a dosage form.

A “Suspo-emulsion” is a formulation in which oil droplets and particles of a water-insoluble drug are dispersed and suspended together in water. A drug or an active may be dissolved in an inert oil, which is then emulsified in the system. If the drug is an oily form, it can be directly emulsified in the system or it can be diluted in an inert oil before emulsification. There is a need that these ingredients are stable when dispersed together in water not only immediately after production, but also after storage under suitable conditions. The solid particles to be suspended can be of a drug substance or of a combination of drugs. Even though, active molecules are suspended in the dosage form, a small portion of drug molecules can be in a molecularly dissolved state and can be considered as a solution. Thus, the active water-insoluble drug molecules are mostly in a suspended state, and a small portion in a dissolved state and oily components are in the emulsified state. This patent does not specify and claim the quantification of drugs in the dissolved, suspended or in emulsified state.

“Novel Formulation” means any new formulation, which is different than the existing formulation.

A “humectant” is a substance, which retains moisture in the formulation or keep thing moist. Humectants are found in moisturizers. Humectants are used in some of the formulation and are selected from the group consisting of sorbitol, propylene glycol, polyethylene glycol, glycerin and mixtures thereof.

We examined the effect of medium chain triglycerides on the evaporation rates of essential oils. A mixture of cinnamon oil, tea tree oil and lemon grass oil (210.1 mg, 175.5 mg and 178.6 mg, respectively) was prepared. About 25 to 30 mg of the mixture was weighed in 10 empty vials to which different amounts of medium chain triglyceride (MCT) were added (0 to 350% of the weight of mixture of essential oils). The samples were weighed over 5 days at intermittent intervals. The weight-loss values were determined. After 9.1 hrs, the weight loss values for the sample without MCT and for the samples with 349% MCT were 8.4% and 1.8%, respectively. There was a qualitative correlation between the weight-loss values and the percent MCT added to each sample throughout the experiment. Overall, it became evident that MCT reduced the evaporation rate of the mixture of essential oil used.

### Examples of Various Formulations

With the following examples, one skilled in the art, can understand and use the present invention. In general, all the formulation contained one or two anthelmintic compounds, a mixture of EOs, medium chain triglyceride, a humectant, surfactants and water.

Composition 1—It contained Albendazole as the active anthelmintic drug. The formulation contained very small percentage of oils. Thus, the water-insoluble active. Albendazole, may have some solubility in oils. But the amount of albendazole dissolved in the oil phase can be considered as insignificant.

TABLE 1
Composition for 100 g naso-suspension formulation

| Ingredient | Amount, w/w |
|---|---|
| Albendazole | 5.0% |
| Tea tree oil | 0.01% |
| Cinnamon oil | 0.01% |
| Lemongrass oil | 0.01% |
| Sorbitol | 1% |
| Medium chain triglyceride | 0.5% |
| Tween 80 | 0.25% |
| Span 80 | 0.1% |
| Water | 93.12% |

The preceding formulation is prepared by mixing the various constituents and stirring. The formulation was homogenized with Polytron 3000 to form a pre-emulsion. Then formulation was then passed through a microfluidizer. A microfluidizer from Microfluidics (Model 110P) was used in these experiments. Particle size distribution (PSD) values were determined after completion of predetermined number of passes. Normally, PSD was determined after 1 to 2 passes. PSD values were determined using Malvern 2000.

Composition 2—It contained Niclosamide as the active anthelmintic drug. The percent of niclosamide can be altered once more in vivo studies are performed.

TABLE 2
Composition for 100 g suspo-emulsion
formulation containing niclosamide.

| Ingredient | Amount, w/w |
|---|---|
| Niclosamide | 10.0% |
| Peppermint oil | 0.05% |
| Grapefruit oil | 0.05% |
| Eucaplyptus oil | 0.05% |
| Propylene glycol | 1.5% |
| Medium chain triglyceride | 0.8% |
| Tween 80 | 1% |
| Span 80 | 0.1% |
| Water | 86.45% |

The procedure to prepare compositions 2 and 3 is similar, which involved dissolution of essential oils in medium chain triglyceride, emulsification followed by an addition of solid water-insoluble drug to form a suspo-emulsion.

Composition 3—It contained Ivermectin as the active anthelmintic drug and three different essential oil dissolved in MCT.

TABLE 3
Composition for 100 g suspo-emulsion
formulation containing ivermectin.

| Ingredient | Amount, w/w |
|---|---|
| Ivermectin | 1.0% |
| Pine oil | 0.05% |
| Thyme oil | 0.05% |
| Oregano oil | 0.05% |
| Propylene glycol | 1.5% |
| Medium chain triglyceride | 0.8% |
| Tween 80 | 0.5% |
| Span 80 | 0.3% |
| Water | 95.75% |

Composition 4—This formulation contained two anthelmintic actives—mebendazole and ivermectin along with three essential oils.

TABLE 4
Composition for 100 g suspo-emulsion formulation containing a
combination of anthelmintic drugs - mebendazole and ivermectin.

| Ingredient | Amount, w/w |
|---|---|
| Mebendazole | 2.5% |
| Ivermectin | 1.0% |
| Eucalyptus oil | 0.05% |
| Peppermint oil | 0.05% |
| Pine oil | 0.05% |
| Propylene glycol | 1.5% |
| Medium chain triglyceride | 1.0% |
| Tween 80 | 0.5% |
| Span 80 | 0.3% |
| Water | 93.05% |

Composition 5—This formulation contained two anthelmintic actives—niclosamide and ivermectin along with three essential oils.

TABLE 5
Composition for 100 g suspo-emulsion formulation
containing a combination of anthelmintic drugs.

| Ingredient | Amount, w/w |
|---|---|
| Niclosamide | 10% |
| Ivermectin | 0.5% |
| Peppermint oil | 0.05% |
| Eucalyptus oil | 0.05% |
| Cinnamon oil | 0.05% |
| Propylene glycol | 1.5% |
| Medium chain triglyceride | 1.0% |
| Tween 80 | 0.5% |
| Span 80 | 0.3% |
| Water | 86.05% |

Particle Size Distribution in Suspo-Emulsion Formulation

In the case of naso-pulmonary drug delivery for systemic absorption, aerosols with small particle size are required to ensure penetration of drug. Particles <3 μm have an approximately 80% chance of reaching the lower airways with 50-60% being deposited in the alveoli. Two batches of drug along with essential oils were prepared. Both the batches were first homogenized and then passed through a microfluidizer. Table 5 lists the particle size distribution (PSD) achieved for a representative suspo-emulsion formulation of ivermectin with essential oils. The particle size distribution is a combination of PSD for the suspended particles and PSD for the oil globules for the emulsion.

TABLE 6
Particle Size Distribution for a Suspo-emulsion formulation.
The particle size values are in microns.

| | | | | | | | Weighted |
|---|---|---|---|---|---|---|---|
| | D (0.1) | D (0.5) | D (0.9) | D (1.0) | D [3, 2] | D [4, 3] | Residual |
| Initial | 0.112 | 0.568 | 2.104 | 20.000 | 0.277 | 0.952 | 3.521 |
| After 3 passes | 0.068 | 0.144 | 0.543 | 7.962 | 0.125 | 0.280 | 2.028 |
| After 5 passes | 0.066 | 0.135 | 0.409 | 7.962 | 0.117 | 0.252 | 1.733 |

Prior to microfluidization, a primary emulsion was formed using Polytron 3000 homogenizer. The D90 values were 2.1 microns. After microfluidization, the D10, D50 and D90 values found to be 66 nm, 135 nm and 409 nm, respectively. These PSD values were satisfactory. No settling of suspended particle forming a cake or breaking of the emulsion were observed. The suspo-emulsion of Ivermectin with essential oils were satisfactory.

Theoretically, in nanosuspensions or in nanoemulsions, one expects the D90 value (90% units are below this value) of particles or globules to be less than 200 nm. Some references define nanosuspension or nanoemulsion dosage forms as those with D90 value of 1 micron or less. Some references define microsuspension or microemulsion dosage forms as those with D90 value of 25 micron or less. In this patent, we have not defined our system as a nanosuspo-emulsion or microsuspo-emulsion. But our intend is to prepare either nanosuspo-emulsion or microsuspo-emulsion dosage forms. The product will be sprayed in the naso-pulmonary cavity and it is important to achieve lower particle size distribution for two reasons. The dissolution rate of the suspended drug is significantly higher for micro- and nano-particles as the surface area for dissolution increases with a reduction in the particle size. Secondly, the particle size affect the reach of particles in the respiratory system. We observed that we could easily produce nanosuspo-emulsion or microsuspo-emulsion products. For convenience, we termed our system as a suspo-emulsion dosage form.

Concluding Remarks

In this patent, we prepared a suspo-emulsion dosage form for naso-pulmonary delivery of drugs. We used a combination of an allopathic drug(s) a and natural products. A suspo-emulsion dosage form of anthelmintic drug(s) such as Ivermectin with essential oils was prepared. The particle size was reduced using a microfluidizer and a D90 value of less than 1 micron was observed. We want to claim the D90 value of less than 23 microns for the formulations. The dosage form is proposed to be used for administration to the nasal cavity and lungs (naso-pulmonary administration). Essential oils can reach up to sinuses where the viruses tend to reside during the first couple of days after invasion into the nasal cavity. The intention of this dosage form is to achieve therapeutically or pharmacologically effective levels or prophylactic levels of actives locally in the naso-pulmonary regions. Any formulation scientist skilled in the art will be able to develop these formulations.

The description of formulations and concept in the present invention has been presented for purposes of illustration, but it is not intended to be exhaustive or limited to the invention in the form disclosed. Many modifications and variations may become apparent to those of ordinary skill in the art without departing from the scope and spirit of the invention. The description in the patent explains the principles of the invention and the practical applications, and to enable others of ordinary skill in the art to understand the invention for various embodiments with various modifications as are suited to the particular use contemplated.

### REFERENCES

1. H. N. Joshi and N. Sharma; Composition of Suspoemulsion formulation of Anthelmintic drugs with essential oils for naso-pulmonary administration; Provisional U.S. Patent Application 63/107,624; EFS ID—40995139; Filed on Oct. 30, 2020.
2. F. R. Formiga, R. Leblanc, J. de S. Reboucas, L. P. Farias, R. N. de Oliveira, and L. Pena, Ivermectin: an award-winning drug with expected antiviral activity against Covid-19, J. Controlled Rel., Oct. 4, 2020.
3. F. Heidary and R. Gharbaghi; Ivermectin: a systematic review from antiviral effects to Covid-19 complementary regimen; The J. of Antibiotics 73:593-602 (2020).
4. G. R. Gandhi, A. B. S. Vasconcelos, G. Hariharan, V. K. da Silva Calisto, G. Jothi, J. deSouza S. Quintans, L. E. Cuevas, N. Narain, L. J. Q. Junior, R. Cipolotti and R. Q. Gurgel: Essential oils and its bioactive compounds modulating cytokines: A systematic review on anti-asthmatic and immunomodulatory properties; Phytomedicine 73: 152854 (July 2020).
5. Reeve and T. P. J. Michael; Antimicrobial and/or antiviral composition and to methods for preparing and administering same; U.S. Pat. No. 9,655,939: May 23, 2017.
6. M. Asif et al., Covid 19 and therapy with essential oils having antiviral, anti-inflammatory, and immunomodulatory properties, Inflammopharmacology, Aug. 14, 2020, pages 1 to 9.
7. L. Caly, J. D. Druce, M. G. Catton, D. A. Jans, and K. M. Wagstaff. The FDA approved drug ivermectin inhibits the replication of SARS-CoV-2 in vitro; Antivir. Res. 178 (2020) 104787.
8. V. D. Schmith, J. J. Zhou and L. R. L. Lohmer; The Approved dose of ivermectin alone is not the ideal dose for the treatment of Covid-19, Clin. Pharmacol. Ther. 108: 762-765 (2020).
9. B. Jermain, P. O. Hanafin, Y. Cao, A. Lifschitz, C. Lanusse and G. G. Rao; Development of a minimal physiologically-based pharmacokinetic model to simulate lung exposure in humans following oral administration of ivermectin for Covid-19 repurposing; J. Pharm. Sci. 109: 3574-3578 (2020).
10. S. Y. Tay. B. R. Ramasamy, D. A. Watson and M. Montoya; Treatment of nasal myiasis with ivermectin irrigation; http://dx.doi.org/10.1136/bcr-2017-224142 (2018).
11. W. Chen, R. A. Mook, Jr., R. T. Premont, and J. Wang; Niclosamide: Beyond an antihelminthic drug; Cellular Signalling 41: 89-96 (2018).
12. N. S. Rejinold, H. Piao, G-W. Jin, G. Choi, and J-H. Choy; Injectable niclosamid nanohybrid as an anti-SARS-CoV-2 Strategy; Colloids surf. B. Biointerfaces 2021 Aug. 24; 208:112063. doi: 10.1016/j.colsurfb.2021.112063.
13. J. Xu. Y. Shi, H. Li, and J. Zhou; Broad Spectrum Antiviral agent niclosamide and its therapeutic potential; ACS Infect. Dis. 6: 909-915 (2020).
14. A. Brunaugh, H. Seo, Z. Warnken, L. Ding, S. H. Seo, and H. D. C. Smyth; Broad-spectrum, patient-adaptable inhaled niclosamide-lysozyme particles are efficacious against coronaviruses in lethal murine infection models; bioRxiv preprint doi:haps://doi.org/10.1101/2020.09.24.310490.

What is claimed:

1. A composition of a suspo-emulsion dosage form for naso-pulmonary administration, comprising: (i) therapeutically effective or prophylactic amount of at least one anthelmintic drug; (ii) therapeutically effective or prophylactic amount of at least one essential oil; (iii) at least one surfactant and (iv) pharmaceutically acceptable excipients; wherein the D90 value of the suspended particles and emulsified globules is less than or equal to 25 microns; wherein at least one anthelmintic drug is ivermectin.

2. A composition of a suspo-emulsion dosage form for naso-pulmonary administration according to claim 1, wherein the anthelmintic compound is selected from the group consisting of ivermectin, albendazole, mebendazole, thiabendazole, fenbendazole, benzimidazole, praziquantel, triclabendazole, flubendazole, abamectin, salicylanilide, nitazoxanide, niclosamide, octadepsipeptides, monepantel, spiroindoles, oxamniquine, piperazine, morantel, pyrantel, levamisole, and tribendimidine.

3. A composition of a suspo-emulsion dosage form for naso-pulmonary administration according to claim 1, wherein the essential oil is selected from the group consisting of tea tree oil, eucalyptus oil, lemongrass oil, cinnamon oil, oils from grapefruit, orange, tangerine, mandarine, lemon, pine, peppermint, lavender, clove, thyme, and oregano.

4. A composition of a suspo-emulsion dosage form for naso-pulmonary administration according to claim 1, wherein the surfactant is of anionic, cationic, non-ionic nature and a mixture thereof.

5. A composition of a suspo-emulsion dosage form for naso-pulmonary administration, comprising: (i) therapeutically effective amounts or prophylactic amounts of at least one anthelmintic drug; (ii) therapeutically effective or prophylactic amount of at least one essential oil; (iii) at least one surfactant and (iv) pharmaceutically acceptable excipients; wherein the D90 value of the suspended particles and emulsified globules is less than or equal to 25 microns; wherein at least one anthelmintic drug is niclosamide.

6. A composition of a suspo-emulsion dosage form for naso-pulmonary administration according to claim 5, wherein the anthelmintic compounds are selected from the group consisting of ivermectin, albendazole, mebendazole, thiabendazole, fenbendazole, benzimidazole, praziquantel, triclabendazole, flubendazole, abamectin, salicylanilide, nitazoxanide, niclosamide, octadepsipeptides, monepantel, spiroindoles, oxamniquine, piperazine, morantel, pyrantel, levamisole, and tribendimidine.

7. A composition of a suspo-emulsion dosage form for naso-pulmonary administration according to claim 5, wherein the essential oil is selected from the group consisting of tea tree oil, eucalyptus oil, lemongrass oil, cinnamon oil, oil from grapefruit, orange, tangerine, mandarine, lemon, pine, peppermint, lavender, clove, thyme, and oregano.

8. A composition of a suspo-emulsion dosage form for naso-pulmonary administration according to claim 5, wherein the surfactant is of anionic, cationic, non-ionic nature and a mixture thereof.

9. A composition of a suspo-emulsion dosage form for naso-pulmonary administration, comprising: (i) therapeutically effective amounts or prophylactic amounts of at least anthelmintic drug; (ii) therapeutically effective or prophylactic amounts of at least two essential oils; (iii) at least one surfactant and (iv) pharmaceutically acceptable excipients; wherein the D90 value of the suspended particles and emulsified globules is less than or equal to 25 microns.

10. A composition of a suspo-emulsion dosage form for naso-pulmonary administration according to claim 9, wherein the anthelmintic compound is selected from the group consisting of ivermectin, albendazole, mebendazole, thiabendazole, fenbendazole, benzimidazole, praziquantel, triclabendazole, flubendazole, abamectin, salicylanilide, nitazoxanide, niclosamide, octadepsipeptides, monepantel, spiroindoles, oxamniquine, piperazine, morantel, pyrantel, levamisole, and tribendimidine.

11. A composition of a suspo-emulsion dosage form for naso-pulmonary administration according to claim 9, wherein the essential oils are selected from the group consisting of tea tree oil, eucalyptus oil, lemongrass oil, cinnamon oil, oil from grapefruit, orange, tangerine, mandarine, lemon, pine, peppermint, lavender, clove, thyme, and oregano.

12. A composition of a suspo-emulsion dosage form for naso-pulmonary administration according to claim 9, wherein the surfactant is of anionic, cationic, non-ionic nature and a mixture thereof.
